(19) United States
(12) Patent Application Publication
(10) Pub. No.: US 2026/0290497 A1
(43) Pub. Date: Sep. 24, 2026

(54) Systems and Methods for Protein Structure Prediction Using Recognition Coverage Functions and Adaptive Conformational Optimization

(71) Applicant: Jonathan Washburn, Austin, TX (US)

(72) Inventor: Jonathan Washburn, Austin, TX (US)

(21) Appl. No.: 19/570,467

(22) Filed: Mar. 18, 2026

Related U.S. Application Data
(60) Provisional application No. 63/774,248, filed on Mar. 19, 2025.

(51) Int. Cl. G16B 15/20

(52) U.S. Cl. CPC G16B 15/20

(57) ABSTRACT

Systems and methods for protein structure prediction are disclosed. In some embodiments, a protein-related input is received and one or more candidate conformations are generated. A nonnegative conformation-associated energy or cost is computed for at least one candidate conformation and transformed using a recognition coverage function. A transformed conformational objective is formed based at least in part on the transformed energy or cost, and the candidate conformation is iteratively updated using the transformed conformational objective to produce one or more predicted protein structures or structure-related prediction results. In some embodiments, the recognition coverage function is defined as F_cov(E, X_opt)=E/(E+X_opt), where E is the nonnegative conformation-associated energy or cost and X_opt is a scaling parameter. The disclosed systems and methods may be implemented in local, networked, cloud-based, or hardware-accelerated environments.

### CROSS-REFERENCE TO RELATED APPLICATIONS

This application claims the benefit of and priority to U.S. Provisional Patent Application No. 63/774,248, filed on Mar. 19, 2025, the entirety of which is hereby incorporated by reference herein.

### BACKGROUND

The present disclosure relates generally to computational biology, structural bioinformatics, and molecular modeling, and more particularly to computational methods and systems for protein structure prediction, conformational optimization, fold analysis, and structure-guided screening across software, hardware, and networked computing environments.

Protein structure prediction remains a difficult computational problem because a protein sequence may correspond to an enormous number of possible conformational states. As protein length, flexibility, and domain complexity increase, the size of the accessible conformational search space grows rapidly, making exhaustive evaluation impractical. Conventional approaches, including molecular dynamics, Monte Carlo sampling, fragment-based sampling, and related simulation or search techniques, can require substantial computational time and resources to identify candidate structures that satisfy geometric, energetic, and biochemical constraints. Even for proteins of moderate length, conformational exploration may involve an extremely large number of candidate states, which can make accurate and efficient structure determination challenging.

Existing protein-folding and structure-prediction approaches also face accuracy limitations. In many implementations, prediction quality depends heavily on assumptions embedded in force fields, scoring functions, templates, restraints, initialization strategies, or other modeling choices. Such approaches may perform unevenly across different protein classes, including multi-domain proteins, flexible proteins, engineered variants, therapeutic proteins, membrane-associated targets, and other structurally complex systems. In addition, scoring and ranking mechanisms that work adequately for some targets may fail to distinguish effectively among competing conformations for other targets, thereby limiting reliability and general applicability.

Another technical challenge arises from convergence and optimization stability. Protein-folding computations frequently involve high-dimensional coordinate spaces, nonlinear objective landscapes, competing local minima, steric constraints, and coupled residue interactions. In such settings, optimization procedures may converge slowly, oscillate, become trapped in undesirable local minima, or expend substantial computation exploring regions of conformational space that do not yield useful structural candidates. Step-size selection, update scheduling, and weighting of local versus global structural contributions can materially affect whether a computational workflow converges efficiently and whether the resulting structures are physically plausible and useful for downstream applications.

Practical deployment constraints further complicate protein structure prediction. Modern research and commercial workflows often require compatibility with existing molecular-modeling frameworks, simulation engines, force-field libraries, and validation pipelines. At the same time, there is increasing demand for implementations that can operate efficiently on heterogeneous computing platforms, including CPUs, GPUs, FPGA-based accelerators, ASIC-based accelerators, edge devices, and cloud-based infrastructures. Conventional approaches may be difficult to adapt cleanly across such environments while preserving acceptable runtime, throughput, reproducibility, and output quality.

There is also a growing need for protein-structure prediction workflows that produce outputs usable beyond a single academic prediction setting. In many commercial, industrial, and biomedical contexts, predicted structures may be used for mutation assessment, enzyme engineering, therapeutic-protein evaluation, target analysis, foldability assessment, instability localization, screening, ranking, or other downstream decision-making processes. These workflows may benefit from computational methods that are more consistent, more scalable, and better suited for integration into broader discovery and engineering pipelines.

Accordingly, there remains a need for improved computational methods and systems for protein structure prediction that reduce computational burden, improve structural prediction quality, provide more stable convergence behavior in complex conformational landscapes, remain compatible with existing molecular-modeling infrastructure, and operate effectively across workstation, accelerator, edge, and cloud computing environments.

### SUMMARY

The present disclosure provides systems and methods for computational protein structure prediction using a recognition coverage function applied to a nonnegative conformation-associated energy or cost. In some embodiments, a candidate protein conformation is evaluated by obtaining or computing a nonnegative energy quantity E associated with the candidate conformation, transforming the energy quantity using a recognition coverage function, forming a transformed conformational objective based on the transformed energy quantity, iteratively updating the candidate conformation using one or more optimization parameters modulated by the transformed energy quantity, and outputting one or more predicted protein structures, ranked candidate structures, confidence values, foldability metrics, instability indicators, or combinations thereof.

In some embodiments, the recognition coverage function is defined as:

$$F\_cov ( E , X\_opt ) = E / ( E + X\_opt )$$

In some embodiments, the scaling parameter is defined as:

$$X\_opt = phi / pi$$

In this disclosure, the terms “phi” and “pi” may be written using ASCII characters (“phi” and “pi”) or using corresponding symbols (φ and π) in mathematical expressions, and the symbols and ASCII forms refer to the same quantities. In embodiments, phi is the golden ratio and pi is the circle constant.

In some embodiments, phi is the golden ratio. In some embodiments, pi is the circle constant. In some embodiments, X_opt is fixed for a prediction workflow. In some embodiments, X_opt is used as a constant scaling value for transforming a nonnegative conformation-associated energy or cost into a bounded quantity that supports stable optimization behavior.

In some embodiments, the transformed conformational objective is formed using one or more local energy terms associated with one or more portions of a candidate conformation. In some embodiments, a local transformed objective is defined as:

$$E\_RCF , i ( C ) = F\_cov ( E\_i ( C ) , X\_opt ) * ( Delta theta\_i ) \hat{} 2$$

where E_i(C) is a local nonnegative energy or cost associated with a candidate conformation C and a selected structural component i, and Delta theta_i represents a structural deviation, angular deviation, torsional deviation, positional deviation, or other deviation quantity associated with the selected structural component. In some embodiments, a total transformed objective is defined as:

$$E\_RCF ( C ) = sum\_i [ F\_cov ( E\_i ( C ) , X\_opt ) * ( Delta theta\_i ) \hat{} 2 ]$$

In some embodiments, the transformed conformational objective is based on local terms, global terms, pairwise terms, residue-wise terms, domain-wise terms, graph-based terms, contact-based terms, or combinations thereof.

In some embodiments, a candidate conformation is iteratively updated according to:

$$alpha\_k = alpha\_ 0 * F\_cov ( E ( C\_k ) , X\_opt )$$

$$C\_ ( k + 1 ) = C\_k - alpha\_k * grad ( E\_RCF ( C\_k ) )$$

where C_k is a candidate conformation at iteration k, alpha_0 is a base update parameter, alpha_k is an iteration-specific update parameter, E(C_k) is a nonnegative conformation-associated energy or cost of the candidate conformation, and grad(E_RCF(C_k)) is a gradient, derivative, directional update, or other optimization quantity associated with the transformed conformational objective. In some embodiments, the transformed quantity produces adaptive step modulation such that update behavior differs between relatively high-energy and relatively low-energy conformational regions. In some embodiments, this supports more stable traversal of a rugged conformational landscape, reduced overshoot near lower-energy regions, and improved convergence behavior. In some embodiments, the bounded recognition coverage transformation provides dynamic-range compression for larger energy or cost values while preserving monotonic ordering over a nonnegative domain, which reduces oscillatory updates near lower-energy regions and reduces wasted computation exploring high-energy regions. In some embodiments, these effects manifest as one or more measurable computational improvements, including reduced iteration count to satisfy stopping conditions, reduced variance in update magnitude across iterations, increased restart success rate, increased acceptance rate under validation threshold bands, improved stability of intermediate conformations (e.g., reduced clash spikes), or combinations thereof.

In some embodiments, the nonnegative conformation-associated energy or cost E is derived from a Recognition Science cost, a recognition-based cost, a conventional force-field energy that has been shifted, rectified, normalized, decomposed, clipped, or otherwise transformed to satisfy nonnegativity, or a hybrid quantity derived from both recognition-based and conventional force-field information. In some embodiments, E is based on one or more of bond terms, angle terms, torsion terms, steric terms, electrostatic terms, solvation terms, contact terms, residue-neighborhood terms, graph-based terms, strain terms, realization-cost terms, or combinations thereof. In some embodiments, the disclosed systems and methods are not limited to a single energy construction technique.

In some embodiments, the present disclosure provides a computational method comprising receiving a protein-related input, generating one or more candidate conformations corresponding to the protein-related input, computing one or more nonnegative conformation-associated energies or costs for the one or more candidate conformations, transforming the one or more energies or costs using the recognition coverage function, forming one or more transformed conformational objectives, iteratively optimizing the one or more candidate conformations using update parameters modulated by the transformed values, evaluating one or more stopping conditions, and outputting one or more predicted structures or related prediction results. In some embodiments, the protein-related input includes an amino-acid sequence, a FASTA-formatted sequence, a partial structure, one or more restraints, one or more template structures, one or more mutation variants, one or more domain definitions, one or more ligand or cofactor indicators, or combinations thereof.

In some embodiments, the present disclosure provides a system comprising one or more processors and one or more memories storing instructions that, when executed by the one or more processors, cause the system to perform operations comprising sequence intake, optional preprocessing, candidate conformation generation, nonnegative energy computation, recognition coverage transformation, transformed objective generation, conformational optimization, convergence checking, validation, ranking, and output generation. In some embodiments, the system includes an input interface, a preprocessing module, an initialization module, an energy evaluation module, a recognition coverage function module, an optimization module, a validation module, a ranking module, a data store, a network interface, and an output module. In some embodiments, the modules are implemented in software, hardware, firmware, or any combination thereof.

In some embodiments, the present disclosure provides a non-transitory computer-readable medium storing instructions that, when executed by one or more processors, cause the one or more processors to perform any of the methods disclosed herein. In some embodiments, the present disclosure provides a cloud-based service configured to receive protein-related input through an application programming interface, a web interface, a job queue, or another network-accessible interface, and to return predicted structures, ranked outputs, confidence values, foldability metrics, structural reports, or combinations thereof. In some embodiments, the present disclosure provides a hardware-accelerated platform implemented using one or more CPUs, GPUs, FPGAs, ASICs, edge devices, servers, clusters, or cloud-computing resources.

In some embodiments, the disclosed systems and methods interoperate with one or more external molecular-modeling or molecular-simulation frameworks. In some embodiments, the disclosed systems and methods interface with one or more force-field frameworks, molecular-dynamics frameworks, structural-biology pipelines, validation systems, or protein-modeling environments. In some embodiments, interoperability is provided with OpenMM, AMBER, CHARMM, or other molecular-modeling infrastructures. In some embodiments, the recognition coverage function is applied as part of a plugin, wrapper, sidecar scoring engine, preprocessing stage, in-loop optimization stage, or post-processing refinement stage.

In some embodiments, the present disclosure provides generalized recognition coverage functions. In some embodiments, a generalized recognition coverage function is defined as:

$$F\_alt ( E , X\_opt , alpha ) = E \hat{} alpha / ( E \hat{} alpha / ( E \hat{} alpha + X\_opt \hat{} alpha )$$

where alpha is a positive parameter. In some embodiments, alpha is selected to control compression behavior, scaling sensitivity, or optimization response. In some embodiments, alpha is fixed. In some embodiments, alpha is selected from a predetermined range. In some embodiments, alpha is selected adaptively. In some embodiments, the generalized recognition coverage function remains bounded and monotone over a nonnegative energy domain.

In some embodiments, the disclosed systems and methods support multiple initialization and search strategies. In some embodiments, candidate conformations are initialized randomly, template-guided, fragment-guided, coarse-grained, backbone-first, multiple-restart, domain-wise, mutation-local, or using another initialization strategy. In some embodiments, optimization is performed using gradient descent, projected gradient descent, coordinate descent, stochastic updates, momentum-based updates, quasi-Newton updates, trust-region updates, beam-search-assisted updates, population-assisted updates, simulated annealing, or hybrid optimization workflows. In some embodiments, conformational search is performed in a coarse-to-fine manner, a multi-stage manner, a domain-wise manner, or a parallelized manner.

In some embodiments, the disclosed systems and methods support search-space reduction and pruning. In some embodiments, pruning is based on contact constraints, residue-neighborhood restrictions, graph constraints, clash filters, torsion-window constraints, domain decomposition, beam truncation, mutation-local restrictions, or other selection criteria. In some embodiments, such pruning reduces effective search burden while preserving candidate conformations that are likely to yield useful predicted structures.

In some embodiments, the disclosed systems and methods support validation, ranking, and downstream use of prediction outputs. In some embodiments, outputs include Cartesian coordinates, backbone coordinates, all-atom coordinates, coarse-grained structures, ranked candidate structures, confidence values, foldability scores, instability maps, misfolding indicators, contact satisfaction values, convergence records, or combinations thereof. In some embodiments, validation and ranking are based on one or more of RMSD, structural consistency, contact recovery, constraint satisfaction, internal stability, consensus across restarts, comparison against reference structures, or other validation metrics.

In some embodiments, the disclosed systems and methods support adaptive feedback. In some embodiments, one or more optimization parameters are updated based on validation loss, convergence performance, structural quality, or benchmark performance. In some embodiments, a base update parameter alpha_0 is adjusted while X_opt remains fixed. In some embodiments, adaptive feedback is performed online, offline, periodically, continuously, protein-class-wise, sequence-length-wise, or according to another control schedule.

In some embodiments, the disclosed systems and methods are used for mutation analysis, enzyme engineering, antibody engineering, therapeutic-protein design, peptide screening, drug discovery, target analysis, personalized medicine, agricultural biotechnology, industrial biotechnology, protein variant ranking, instability localization, or other structure-guided applications. In some embodiments, the output of the disclosed systems and methods is used to identify relatively stable conformations, rank sequence variants, detect misfolding-prone regions, prioritize candidate molecules, guide experimental screening, or support downstream engineering decisions.

In some embodiments, folding is additionally or alternatively modeled using one or more trajectory-based, strain-based, realization-based, or codon-derived representations. In some embodiments, a candidate structure is evaluated using a trajectory strain, a local strain, a realization cost, a codon-derived representation, a Q_6 representation, or combinations thereof. In some embodiments, such representations are used as optional embodiments for generating, modifying, constraining, or evaluating the nonnegative conformation-associated energy or cost E. In some embodiments, folding is treated as a minimization of trajectory strain or realization cost over a codon-derived or qualia-derived representation of a protein sequence.

Accordingly, the present disclosure provides a family of computational protein structure prediction systems and methods in which a recognition coverage function is used to transform a nonnegative conformation-associated energy or cost into a bounded quantity that supports adaptive conformational optimization, dynamic-range compression, improved convergence behavior, interoperability with existing molecular-modeling infrastructure, and deployment across software, hardware, accelerator, edge, and cloud environments.

### BRIEF DESCRIPTION OF THE DRAWINGS

FIG. 1 illustrates an example system for protein structure prediction using recognition coverage functions and adaptive conformational optimization.

FIG. 2 illustrates an example method for receiving a protein-related input, generating one or more candidate conformations, computing one or more nonnegative conformation-associated energies or costs, transforming the one or more energies or costs using a recognition coverage function, iteratively optimizing the one or more candidate conformations, and outputting one or more prediction results.

FIG. 3 illustrates an example recognition coverage function relationship between a nonnegative conformation-associated energy or cost E and a bounded transformed quantity F_cov(E, X_opt).

FIG. 4 illustrates an example transformed conformational objective based on one or more local, global, residue-wise, pairwise, graph-based, or hybrid energy terms weighted using a recognition coverage function.

FIG. 5 illustrates an example adaptive update schedule in which an iteration-specific update parameter is modulated using a recognition coverage function.

FIG. 6 illustrates an example candidate conformation representation including one or more backbone, side-chain, residue-wise, graph-based, coordinate-based, or hybrid structural representations.

FIG. 7 illustrates an example energy evaluation architecture in which one or more recognition-based costs, conventional force-field energies, transformed force-field energies, hybrid energy terms, or combinations thereof are used to form a nonnegative conformation-associated energy or cost.

FIG. 8 illustrates an example optimization architecture including one or more gradient-based, coordinate-based, projected, stochastic, momentum-based, quasi-Newton, trust-region, beam-assisted, population-assisted, or hybrid update processes.

FIG. 9 illustrates an example validation, ranking, and output-generation workflow for producing one or more predicted structures, ranked candidate structures, confidence values, foldability metrics, instability indicators, or related prediction results.

FIG. 10 illustrates an example implementation architecture for executing the disclosed protein structure prediction workflow using one or more local, networked, cloud-based, or hardware-accelerated computing environments.

FIG. 11 illustrates an example mutation-analysis, protein-design, or structure-guided screening workflow using one or more predicted protein structures or related outputs.

FIG. 12 illustrates an example optional codon-derived, trajectory-based, strain-based, realization-cost-based, or Q_6-based embodiment for evaluating or constraining a protein-folding process.

### DETAILED DESCRIPTION

In some embodiments, the various energy constructions and optional embodiments described herein (including recognition-based costs, conventional force-field energies, transformed force-field energies, and hybrid combinations) are alternative implementations under a single inventive concept in which a nonnegative conformation-associated energy or cost is transformed using a recognition coverage function and is used to form and/or modulate a conformational optimization process that produces predicted structures and related outputs.

As used herein, “recognition coverage function” refers to a bounded, monotone transform that maps a nonnegative conformation-associated energy or cost to a value in a bounded range and is usable as a weight, a step modulator, or both. In embodiments, the recognition coverage function is F_cov(E, X_opt)=E/(E+X_opt), where E≥0 and X_opt>0.

Nonnegative conformation-associated energy or cost. In embodiments, the “nonnegative conformation-associated energy or cost” E is obtained by computing one or more raw energy or cost terms and applying a nonnegativity transform selected from shifting by an offset, rectification, clipping, normalization, decomposition into nonnegative components, or combinations thereof, such that the resulting E satisfies E≥0 for conformations evaluated in the workflow. In embodiments, the offset is selected so that typical low-energy conformations remain nonnegative after shifting.

Recognition-based cost (non-limiting examples). In embodiments, a recognition-based cost includes one or more nonnegative mismatch quantities computed between a candidate conformation and one or more structural constraints, contact constraints, neighborhood constraints, learned recognition constraints, template-derived constraints, or combinations thereof. In embodiments, the mismatch quantities are aggregated as a scalar energy or maintained as a set of local values E_i(C) associated with residues, residue pairs, torsion groups, coordinate groups, domains, or graph neighborhoods.

Recognition-cost construction example (contact mismatch). In one implementation, a recognition-based cost is computed from a predicted contact map derived from a candidate conformation C and a target contact prior P derived from the protein-related input (e.g., sequence-derived constraints, template-derived constraints, learned recognition constraints, or combinations thereof). For residue pairs (u, v), a contact indicator I_C(u, v) is computed from an inter-residue distance d_C(u, v) using a threshold d0 (e.g., I_C(u, v)=1 when d_C(u, v)≤d0 and otherwise 0), and a mismatch term is computed as m(u, v)=|I_C(u, v)−P(u, v)|. In embodiments, the recognition-based cost is aggregated as E_rec(C)=Σ_{u, v} w(u, v)·m(u, v), where weights w(u, v) optionally emphasize long-range contacts, domain-interface contacts, or other prioritized interaction subsets.

Local energy bank example. In embodiments, local values E_i(C) are maintained for torsion groups, residue neighborhoods, residue pairs, contact groups, coordinate groups, domains, or graph neighborhoods and are used to compute a transformed conformational objective using recognition coverage weighting, including E_RCF(C)=Σ_i F_cov(E_i(C), X_opt)·(Δθ_i)\hat{ }2, where Δθ_i represents a torsion-angle deviation, coordinate deviation, or other local deviation quantity associated with the i-th local group.

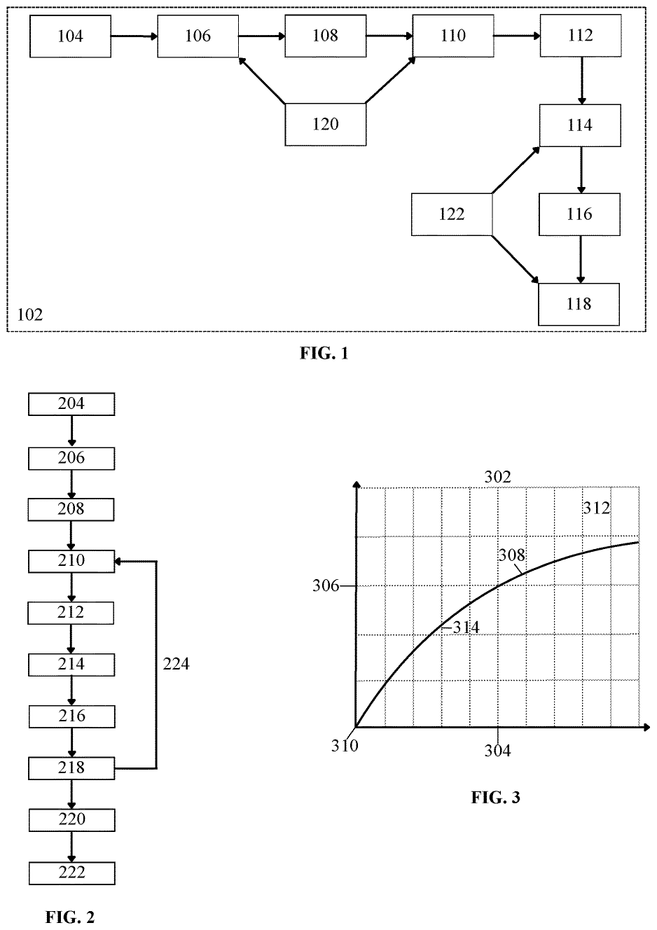

FIG. 1 illustrates an example protein structure prediction platform 102 configured to receive protein-related input, generate one or more candidate conformations, compute one or more nonnegative conformation-associated energies or costs, transform the one or more energies or costs using a recognition coverage function, iteratively optimize the one or more candidate conformations, and output one or more protein structure prediction results. In the illustrated embodiment, the platform 102 includes an input interface 104, a preprocessing engine 106, a candidate conformation generator 108, an energy evaluation engine 110, a recognition coverage transformation engine 112, an optimization engine 114, a validation and ranking engine 116, an output generation engine 118, a data store 120, and a network interface 122. In some embodiments, the platform 102 is implemented on one or more processors executing instructions stored in memory. In some embodiments, one or more of the illustrated elements are implemented in software, hardware, firmware, or combinations thereof. In some embodiments, one or more illustrated elements are combined, separated, distributed, virtualized, replicated, or omitted.

The input interface 104 is configured to receive one or more protein-related inputs. In some embodiments, the protein-related input includes an amino-acid sequence, a FASTA-formatted sequence, a partial structure, a template structure, one or more structural restraints, one or more contact constraints, one or more mutation definitions, one or more domain definitions, one or more ligand or cofactor identifiers, one or more user-selected optimization settings, or combinations thereof. The preprocessing engine 106 is configured to validate, normalize, reformat, annotate, segment, or otherwise prepare the input for downstream structure prediction operations. In some embodiments, the preprocessing engine 106 performs sequence validation, residue normalization, chain segmentation, terminal handling, missing-residue handling, disulfide identification, protonation-state assignment, domain decomposition, graph construction, contact-prior generation, or combinations thereof.

The candidate conformation generator 108 is configured to generate one or more initial candidate conformations responsive to the received input. In some embodiments, the candidate conformation generator 108 produces random initial conformations, fragment-guided conformations, template-guided conformations, coarse-grained conformations, backbone-first conformations, mutation-local conformations, domain-wise conformations, or multiple restart conformations. The energy evaluation engine 110 is configured to compute one or more nonnegative conformation-associated energies or costs for each candidate conformation. The recognition coverage transformation engine 112 is configured to transform the one or more nonnegative energies or costs into one or more bounded transformed quantities using a recognition coverage function. The optimization engine 114 is configured to update one or more candidate conformations using one or more transformed quantities, gradients, directional updates, search policies, or other optimization parameters. The validation and ranking engine 116 is configured to evaluate one or more predicted structures, compare candidate structures, rank candidate structures, and select one or more output structures. The output generation engine 118 is configured to generate output coordinates, structure files, confidence values, foldability metrics, instability indicators, reports, maps, rankings, or other prediction results. The data store 120 is configured to store input data, intermediate states, model data, parameter values, validation records, benchmark records, output data, or combinations thereof. The network interface 122 is configured to support communication with one or more remote devices, networks, application programming interfaces, job queues, storage services, or cloud resources.

In some embodiments, the platform 102 executes a protein structure prediction workflow in which one or more candidate conformations are iteratively refined until one or more stopping conditions are satisfied. In some embodiments, the stopping conditions include a threshold objective value, a threshold objective-improvement value, a threshold gradient norm, a threshold update norm, a threshold confidence value, a threshold structural-stability value, a convergence window condition, an iteration budget, a time budget, or combinations thereof. In some embodiments, the platform 102 operates in a single-stage mode, a multi-stage mode, a coarse-to-fine mode, a restart-based mode, a domain-wise mode, a parallelized mode, a batched mode, or combinations thereof.

FIG. 2 illustrates an example method for receiving a protein-related input, generating one or more candidate conformations, computing one or more nonnegative conformation-associated energies or costs, transforming the one or more energies or costs using a recognition coverage function, iteratively optimizing the one or more candidate conformations, and outputting one or more prediction results. In the illustrated embodiment, the method includes receiving a protein-related input at step 204, preprocessing the protein-related input at step 206, generating one or more candidate conformations at step 208, computing one or more nonnegative conformation-associated energies or costs at step 210, transforming the one or more energies or costs using a recognition coverage function at step 212, forming one or more transformed conformational objectives at step 214, updating one or more candidate conformations at step 216, evaluating one or more stopping conditions at step 218, validating and ranking one or more candidate structures at step 220, and outputting one or more prediction results at step 222. In some embodiments, the method includes a refinement or restart loop 224 in which one or more earlier steps are repeated using modified candidate conformations, modified update parameters, modified constraints, modified resolution levels, or modified search regions.

At step 204, protein-related input is received. In some embodiments, the received input includes a protein sequence and no template structure. In some embodiments, the received input includes a protein sequence and one or more template structures. In some embodiments, the received input includes one or more mutation definitions that identify one or more residue substitutions, insertions, deletions, truncations, or combinations thereof. At step 206, the input is preprocessed to prepare the prediction workflow. In some embodiments, preprocessing includes chain identification, residue cleaning, residue numbering normalization, residue feature extraction, graph generation, or contact-prior generation. At step 208, one or more initial candidate conformations are generated. In some embodiments, a plurality of initial candidate conformations is generated so that downstream optimization may be performed on multiple candidate structures in parallel or in sequence.

At step 210, one or more nonnegative conformation-associated energies or costs are computed for one or more candidate conformations. In some embodiments, the nonnegative conformation-associated energy or cost is represented by E. In some embodiments, E is computed from one or more raw energy or cost terms selected from a force-field energy, a transformed force-field energy, a recognition-based cost, a hybrid energy term, or combinations thereof. In some embodiments, one or more raw terms are converted to a nonnegative quantity by applying a nonnegativity transform selected from shifting by an offset, rectification using a max(0, ·) operation, clipping to a nonnegative range, normalization to a nonnegative range, decomposition into nonnegative components, or combinations thereof, thereby producing E≥0. In some embodiments, E is a scalar value associated with an entire candidate conformation. In some embodiments, E is a vector, set, or collection of local values E_i(C) associated with residues, residue pairs, coordinate groups, torsion groups, domains, contact groups, graph neighborhoods, or combinations thereof. At step 212, the one or more nonnegative energies or costs are transformed using a recognition coverage function to produce one or more bounded transformed quantities. At step 214, one or more transformed conformational objectives are formed using the transformed quantities as weights, modulating factors, or both. At step 216, one or more candidate conformations are updated using one or more transformed objective values, gradients or derivative quantities, directional updates, or control parameters. At step 218, one or more stopping conditions are evaluated. If the stopping conditions are not satisfied, the method returns to step 210, step 212, step 214, or step 216 through the loop 224. If the stopping conditions are satisfied, the method proceeds to step 220 and step 222.

In some embodiments, step 220 includes validating candidate structures using one or more structural or computational metrics. In some embodiments, the one or more metrics include RMSD, structural consistency, contact satisfaction, steric validity, internal stability, foldability score, consensus across restarts, agreement with one or more restraints, agreement with one or more reference structures, or combinations thereof. In some embodiments, step 222 includes outputting one or more Cartesian coordinate files, one or more backbone-only coordinate files, one or more all-atom coordinate files, one or more ranked candidate sets, one or more confidence values, one or more foldability scores, one or more misfolding indicators, one or more instability maps, one or more mutation rankings, one or more engineering recommendations, or combinations thereof.

In an example run, the platform receives an amino-acid sequence in FASTA format for a protein of length N and generates M initial candidate conformations C_0\hat{ }(1) . . . C_0\hat{ }(M) using a fragment-guided initialization or a random initialization. For each candidate conformation C_k, the platform computes one or more local raw energy terms for torsion groups i and converts the local raw terms to nonnegative values using a shift-and-rectify transform, thereby producing local nonnegative values E_i(C_k). In embodiments, E_i(C_k)=max(0, E_i_raw(C_k)+b), where b is an offset selected so that typical low-energy torsions remain nonnegative after shifting. The platform computes a bounded transformed quantity for each local term as w_i=F_cov(E_i(C_k), X_opt) and forms a transformed conformational objective E_RCF(C_k)=Σ_i w_i (Δθ_i)\hat{ }2, where Δθ_i represents a torsion-angle deviation for torsion group i.

In embodiments, the platform computes an iteration-specific update parameter α_k based on the bounded transformed quantity such that α_k=α_0 F_cov(E(C_k), X_opt) or α_k=α_0 F_cov(E_RCF(C_k), X_opt), and updates the conformation according to C_(k+1)=C_k−α_k G_k, where G_k is a gradient, derivative, or directional update quantity derived from the transformed conformational objective. In embodiments, the platform performs multiple restarts and retains one or more best-performing conformations according to the transformed conformational objective and one or more validation metrics.

In embodiments, optimization stops when at least one stopping condition is satisfied, including:
(i) an objective improvement condition in which |E_RCF(C_(k+1))−E_RCF(C_k)|<ε for W consecutive iterations, (ii) a gradient or directional-update magnitude condition in which ∥G_k∥<γ, or (iii) an iteration budget K_max or time budget is reached. In some embodiments, ε is selected from a range of 10\hat{ }−6 to 10\hat{ }−2 (normalized objective units) and W is selected from 5 to 200 iterations. In some embodiments, γ is selected from a range of 10\hat{ }−6 to 10\hat{ }−2 (normalized gradient/update units). In embodiments, after optimization the platform computes one or more validation metrics selected from RMSD (when a reference is available), contact satisfaction, clash score, steric validity, internal stability, foldability score, or consensus across restarts, and applies a decision rule that accepts a candidate structure when at least one validation metric is within an acceptance threshold band and otherwise performs a restart using a modified initialization, modified constraints, modified resolution level, modified α_0, or combinations thereof. In some embodiments, the acceptance threshold band comprises one or more of RMSD ≤T_RMSD (when a reference is available), clash score≤T_clash, and contact satisfaction≥T_contact, where each threshold is selected from a predetermined range and optionally varies by target class, protein length, or workflow stage.

FIG. 3 illustrates an example recognition coverage function relationship 302 between a nonnegative conformation-associated energy or cost E and a bounded transformed quantity F_cov(E, X_opt). In the illustrated embodiment, an energy axis 304 corresponds to nonnegative energy or cost values, and a transformed-value axis 306 corresponds to values of the recognition coverage function. A recognition coverage curve 308 increases monotonically from an origin region 310 toward a saturation region 312. A scaling location 314 corresponds to X_opt. In some embodiments, the recognition coverage function is defined as:

$$F\_cov ( E , X\_opt ) = E / ( E + X\_opt )$$

In some embodiments, the scaling parameter is defined as:

$$X\_opt = phi / pi$$

In some embodiments, phi is the golden ratio and pi is the circle constant. In some embodiments, X_opt is fixed for a protein prediction workflow. In some embodiments, X_opt is fixed across multiple proteins. In some embodiments, X_opt is fixed within a model version, deployment profile, benchmark profile, or optimization profile.

Because E is nonnegative, the recognition coverage curve 308 maps the energy or cost domain into a bounded range. In some embodiments, F_cov(0, X_opt)=0. In some embodiments, F_cov(E, X_opt) approaches 1 as E increases. In some embodiments, the recognition coverage curve 308 provides dynamic-range compression for large energy or cost values while preserving monotonic ordering over a nonnegative domain. In some embodiments, the recognition coverage curve 308 supports more stable optimization behavior by modulating the effect of energy values in different regions of a conformational landscape. In some embodiments, the scaling location 314 corresponds to a region in which response behavior transitions between lower-energy and higher-energy portions of the energy domain.

In some embodiments, a generalized recognition coverage function is also used. In some embodiments, the generalized recognition coverage function is defined as:

$$F\_alt ( E , X\_opt , alpha ) = E \hat{} alpha / ( E \hat{} alpha + X\_opt \hat{} alpha )$$

where alpha is a positive parameter. In some embodiments, alpha is selected to alter compression behavior, response sensitivity, or scaling behavior. In some embodiments, alpha is fixed. In some embodiments, alpha is selected from a predetermined range. In some embodiments, alpha is selected adaptively. In some embodiments, the generalized recognition coverage function is bounded and monotone over a nonnegative energy domain.

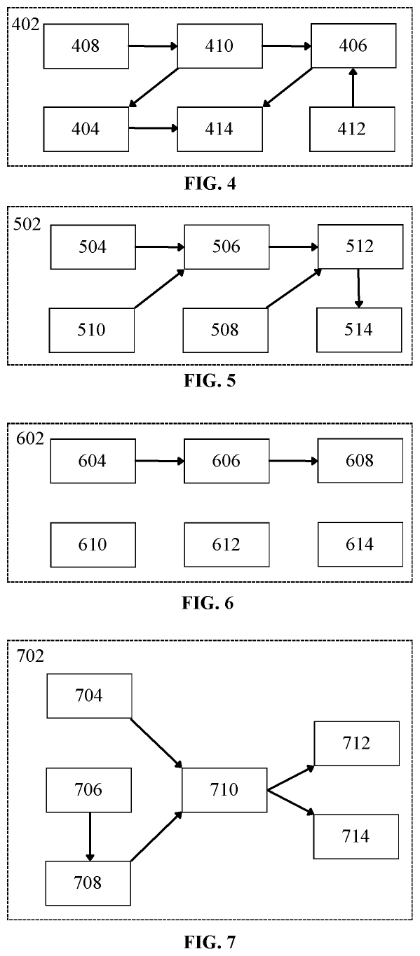

FIG. 4 illustrates an example transformed conformational objective relationship 402 based on one or more weighted structural contributions. In the illustrated embodiment, the relationship 402 includes an untransformed contribution path 404, a transformed contribution path 406, a local structural component 408, a local energy term 410, a local deviation term 412, and a total transformed objective output 414. In some embodiments, the local structural component 408 corresponds to a residue, a residue pair, a torsional component, a coordinate group, a graph neighborhood, a contact group, a domain segment, or another structural portion of a candidate conformation. In some embodiments, the local energy term 410 is represented as E_i(C), where C denotes a candidate conformation. In some embodiments, the local deviation term 412 is represented as Delta theta_i and corresponds to an angular deviation, torsional deviation, positional deviation, distance deviation, strain quantity, realization quantity, or other deviation quantity.

In some embodiments, a local transformed objective contribution is defined as:

$$E\_RCF , i ( C ) = F\_cov ( E\_i ( C ) , X\_opt ) * ( Delta theta\_i ) \hat{} 2$$

In some embodiments, a total transformed objective is defined as:

$$E\_RCF ( C ) = sum\_i [ F\_cov ( E\_i ( C ) , X\_opt ) * ( Delta theta\_i ) \hat{} 2 ]$$

In some embodiments, the total transformed objective output 414 is computed by aggregating a plurality of local transformed contributions. In some embodiments, the transformed contribution path 406 reduces the dominance of relatively large local energy values by converting each local energy value into a bounded weight prior to combination with the local deviation term 412. In some embodiments, the transformed contribution path 406 yields a transformed objective that is less sensitive to raw energy magnitude and more suitable for adaptive conformational optimization in a rugged landscape.

The local energy term 410 may be constructed in various ways. In some embodiments, the local energy term 410 includes one or more bond terms, bond-angle terms, torsion terms, steric terms, electrostatic terms, solvation terms, hydrogen-bond terms, contact terms, graph-based terms, residue-neighborhood terms, strain terms, realization-cost terms, or combinations thereof. In some embodiments, the local energy term 410 is derived from a Recognition Science cost. In some embodiments, the local energy term 410 is derived from a conventional force-field energy that has been shifted, rectified, normalized, decomposed, clipped, or otherwise transformed so that the resulting value is nonnegative. In some embodiments, the local energy term 410 is a hybrid value derived from both recognition-based and conventional force-field information. In some embodiments, the transformed conformational objective relationship 402 is applied to local terms, global terms, pairwise terms, residue-wise terms, domain-wise terms, graph-based terms, contact-based terms, or combinations thereof.

FIG. 5 illustrates an example adaptive update relationship 502 in which an iteration-specific update parameter is modulated using a recognition coverage function. In the illustrated embodiment, the adaptive update relationship 502 includes a base update parameter 504, an iteration-specific update parameter 506, a candidate conformation state 508, an energy evaluation input 510, a transformed update control 512, and an updated conformation output 514.

In some embodiments, the iteration-specific update parameter 506 is defined as:

$$alpha\_k = alpha\_ 0 * F\_cov ( E ( C\_k ) , X\_opt )$$

where alpha_0 corresponds to the base update parameter 504, C_k corresponds to the candidate conformation state 508 at iteration k, and E(C_k) corresponds to the energy evaluation input 510.

In some embodiments, an updated candidate conformation is computed according to:

$$C\_ ( k + 1 ) = C\_k - alpha\_k * grad ( E\_RCF ( C\_k ) )$$

where grad(E_RCF(C_k)) corresponds to a gradient, derivative, directional update, or other optimization quantity used to modify the candidate conformation state 508.

In some embodiments, the transformed update control 512 causes relatively different update behavior in different energy regions. For example, in some embodiments, the iteration-specific update parameter 506 is smaller in lower-energy regions and larger in higher-energy regions, thereby reducing overshoot near lower-energy regions and supporting stronger movement through less favorable regions of the conformational landscape. In some embodiments, the adaptive update relationship 502 acts as a natural annealing or step-modulation mechanism. In some embodiments, the base update parameter 504 is fixed. In some embodiments, the base update parameter 504 is selected per protein, per target class, per sequence length range, per deployment environment, or per model version.

The adaptive update relationship 502 is not limited to a single optimization technique. In some embodiments, the iteration-specific update parameter 506 is used in gradient descent, projected gradient descent, stochastic gradient descent, momentum-based optimization, adaptive-moment optimization, quasi-Newton optimization, trust-region optimization, coordinate descent, beam-assisted local refinement, population-assisted local refinement, simulated annealing, or hybrid update workflows. In some embodiments, different update parameters are assigned to different residues, different domains, different coordinate groups, different torsion groups, or different objective components.

FIG. 6 illustrates an example candidate conformation representation 602 for a protein-related input. In the illustrated embodiment, the representation 602 includes a sequence representation 604, a backbone representation 606, a side-chain representation 608, a residue graph representation 610, a coordinate representation 612, and a domain segmentation representation 614. The sequence representation 604 may correspond to an amino-acid sequence, peptide sequence, or chain-wise sequence input. The backbone representation 606 may correspond to one or more backbone atoms, one or more backbone coordinates, one or more torsion-angle sequences, or one or more backbone segments. The side-chain representation 608 may correspond to one or more side-chain states, one or more rotamer assignments, or one or more side-chain coordinate groups. The residue graph representation 610 may correspond to nodes representing residues and edges representing adjacency, contact likelihood, covalent linkage, domain linkage, or other structural relationships. The coordinate representation 612 may correspond to Cartesian coordinates, internal coordinates, relative coordinates, reduced coordinates, or multiresolution coordinates. The domain segmentation representation 614 may correspond to one or more domains, subdomains, motifs, loops, or structured regions.

In some embodiments, the candidate conformation representation 602 includes only one of the illustrated representations. In some embodiments, the candidate conformation representation 602 includes multiple representations used together during different stages of a prediction workflow. In some embodiments, a coarse-grained representation is used in an early stage and a more detailed coordinate representation is used in a later stage. In some embodiments, the residue graph representation 610 is used to define candidate contact regions, local update neighborhoods, or pruning regions. In some embodiments, the domain segmentation representation 614 is used to perform domain-wise initialization, domain-wise optimization, or domain-wise refinement. In some embodiments, the candidate conformation representation 602 is stored, updated, or translated among multiple internal forms during a protein prediction workflow.

FIG. 7 illustrates an example energy evaluation architecture 702 for computing one or more nonnegative conformation-associated energies or costs. In the illustrated embodiment, the architecture 702 includes a recognition-based energy path 704, a conventional force-field path 706, a transformed force-field path 708, a hybrid energy combination path 710, a nonnegative energy output 712, and an optional local energy bank 714. In some embodiments, the recognition-based energy path 704 computes one or more recognition-based costs associated with a candidate conformation. In some embodiments, the conventional force-field path 706 computes one or more energies using a molecular-modeling framework, force-field library, or simulation engine. In some embodiments, the transformed force-field path 708 converts one or more conventional energies into one or more nonnegative values by shifting, rectifying, clipping, offsetting, normalizing, decomposing, or otherwise transforming the energies. In some embodiments, the hybrid energy combination path 710 combines recognition-based information and force-field-derived information into a shared nonnegative energy output 712.

The optional local energy bank 714 may store one or more residue-wise, pairwise, domain-wise, graph-based, contact-based, or coordinate-group-specific values. In some embodiments, the local energy bank 714 stores values E_i(C) used to compute one or more transformed local objective contributions. In some embodiments, the nonnegative energy output 712 corresponds to a scalar value E(C) used for update modulation, while the local energy bank 714 stores local values used for transformed-objective construction. In some embodiments, the energy evaluation architecture 702 interoperates with OpenMM, AMBER, CHARMM, or other modeling infrastructure. In some embodiments, the energy evaluation architecture 702 is configured as a plugin, wrapper, sidecar scoring engine, preprocessing stage, in-loop evaluation engine, or post-processing refinement engine.

In some embodiments, the nonnegative energy output 712 is broad enough to encompass multiple implementation families. In some embodiments, E is an RS-native nonnegative recognition-cost aggregate. In some embodiments, E is a nonnegative transformed force-field energy. In some embodiments, E is a hybrid quantity derived from both recognition-based and force-field-derived information. In some embodiments, this broad construction allows the disclosed systems and methods to operate in theory-native workflows, conventional-force-field workflows, and hybrid workflows without requiring a change in the overall prediction structure.

FIG. 8 illustrates an example optimization architecture 802 for updating one or more candidate conformations. In the illustrated embodiment, the optimization architecture 802 includes a gradient-based update path 804, a coordinate-based update path 806, a projected update path 808, a stochastic update path 810, a momentum-based update path 812, a quasi-Newton update path 814, a trust-region update path 816, a beam-assisted refinement path 818, a population-assisted refinement path 820, and an updated candidate output 822. In some embodiments, the optimization architecture 802 selects one update path for a given workflow. In some embodiments, the optimization architecture 802 transitions between multiple update paths during different stages of a workflow. In some embodiments, the optimization architecture 802 uses one update path for coarse exploration and another update path for fine refinement.

The updated candidate output 822 may correspond to a single updated candidate conformation, a batch of updated candidate conformations, a ranked subset of candidate conformations, or a refined candidate population. In some embodiments, one or more of the update paths use transformed objective values generated using the recognition coverage function. In some embodiments, one or more update paths use the iteration-specific update parameter defined by alpha_k=alpha_0*F_cov(E(C_k), X_opt). In some embodiments, one or more update paths use local gradients, global gradients, directional derivatives, estimated curvature information, trust-region constraints, projection constraints, or candidate-selection policies. In some embodiments, the optimization architecture 802 performs a coarse-to-fine search, a restart-based search, a domain-wise search, a graph-neighborhood search, a mutation-local search, a contact-restricted search, or combinations thereof.

In some embodiments, the optimization architecture 802 also performs pruning or search-space reduction. In some embodiments, pruning is based on contact constraints, residue-neighborhood restrictions, clash filters, graph restrictions, torsion-window restrictions, domain decomposition, beam truncation, mutation-local restrictions, contact-budget constraints, or combinations thereof. In some embodiments, such pruning reduces the effective search burden while preserving candidate structures that remain likely to yield favorable or useful outputs. In some embodiments, the optimization architecture 802 uses one or more native-contact targets, local-neighborhood bounds, or other structural restrictions to focus computation on a reduced conformational region.

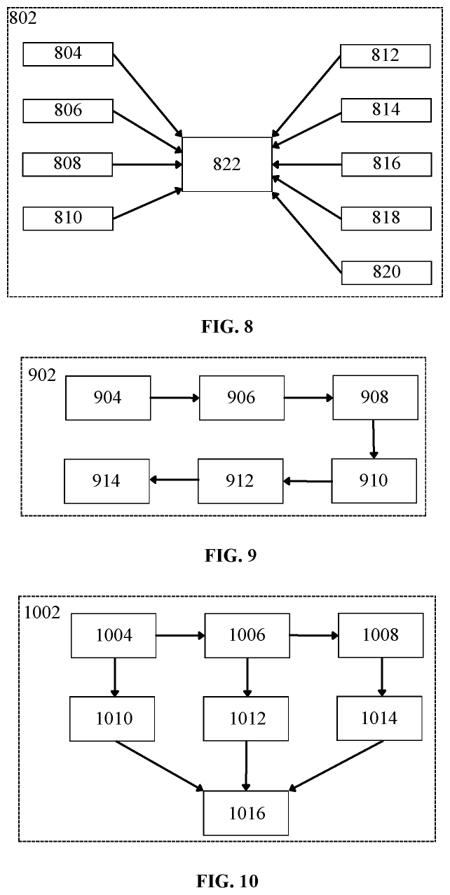

FIG. 9 illustrates an example validation, ranking, and output-generation architecture 902. In the illustrated embodiment, the architecture 902 includes a validation input 904, a structural metric engine 906, a confidence and foldability engine 908, a ranking engine 910, a report generation engine 912, and an output package 914. The validation input 904 may correspond to one or more candidate conformations received from the optimization workflow. The structural metric engine 906 is configured to compute one or more structural or computational metrics, including RMSD, structural consistency, contact recovery, clash information, restraint satisfaction, internal stability, consensus across restarts, agreement with one or more references, or combinations thereof. The confidence and foldability engine 908 is configured to produce one or more confidence values, foldability scores, instability indicators, misfolding-prone region indicators, residue-wise risk maps, domain-wise risk maps, or combinations thereof. The ranking engine 910 is configured to rank candidate structures using one or more metrics, scores, or combined values. The report generation engine 912 is configured to create one or more structure files, reports, maps, rankings, recommendation summaries, or other outputs. The output package 914 may include one or more coordinate outputs, one or more all-atom or backbone models, one or more confidence annotations, one or more ranked candidate lists, one or more engineering recommendations, one or more mutation rankings, or combinations thereof.

In some embodiments, the ranking engine 910 ranks candidate structures according to transformed objective value, validation score, confidence score, consensus behavior, contact satisfaction, structural plausibility, local instability profile, or combinations thereof. In some embodiments, the output package 914 is formatted for direct downstream use in structure-guided screening, enzyme engineering, therapeutic-protein evaluation, mutation analysis, drug discovery, personalized medicine, or agricultural or industrial biotechnology workflows. In some embodiments, the validation, ranking, and output-generation architecture 902 stores provenance, benchmark, or reproducibility records so that prediction runs can be repeated, compared, or audited.

FIG. 10 illustrates an example implementation architecture 1002 for executing the disclosed protein structure prediction workflow using one or more local, networked, cloud-based, or hardware-accelerated environments. In the illustrated embodiment, the implementation architecture 1002 includes a local compute environment 1004, a server environment 1006, a cloud execution environment 1008, a GPU acceleration path 1010, an FPGA acceleration path 1012, an ASIC acceleration path 1014, and an interface layer 1016. In some embodiments, the local compute environment 1004 corresponds to a workstation, laptop, local server, or edge device. In some embodiments, the server environment 1006 corresponds to an on-premises server, cluster node, or managed compute node. In some embodiments, the cloud execution environment 1008 corresponds to a remote compute service configured to receive one or more protein-related inputs through an application programming interface, web interface, job queue, or network service. In some embodiments, the interface layer 1016 supports communication among multiple execution environments, among local and remote resources, or among one or more internal software components and one or more external modeling frameworks.

The GPU acceleration path 1010 may be used for batched candidate evaluation, gradient computation, transformed-objective evaluation, coordinate updates, or other parallelizable operations. The FPGA acceleration path 1012 may be used for low-power on-site screening, fixed-point transformation, streaming evaluation, or hardware-efficient update workflows. The ASIC acceleration path 1014 may be used for dedicated transformed-energy computation, dedicated optimization datapaths, or high-throughput candidate scoring. In some embodiments, the implementation architecture 1002 supports checkpointing, restart, asynchronous job execution, batched execution, distributed candidate pools, domain-parallel processing, sharded ranking, or combinations thereof.

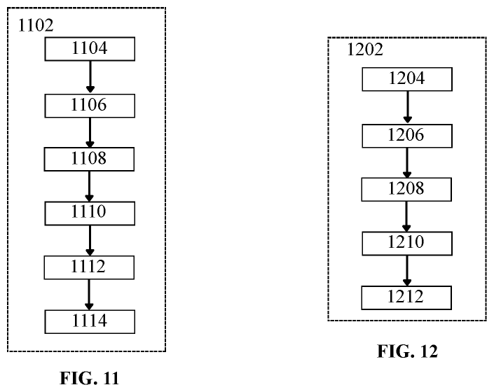

FIG. 11 illustrates an example mutation-analysis, protein-design, or structure-guided screening workflow 1102. In the illustrated embodiment, the workflow 1102 includes a target sequence input 1104, a variant generation stage 1106, a prediction stage 1108, an instability localization stage 1110, a variant ranking stage 1112, and an engineering or screening output stage 1114. The target sequence input 1104 may correspond to a wild-type protein sequence, a known therapeutic protein sequence, a candidate enzyme sequence, a peptide sequence, or another protein-related input. The variant generation stage 1106 may generate one or more substitutions, insertions, deletions, truncations, synonymous variants, nonsynonymous variants, motif edits, domain edits, or combinations thereof. The prediction stage 1108 may execute the disclosed protein structure prediction workflow for each generated variant. The instability localization stage 1110 may identify one or more unstable residues, unstable motifs, misfolding-prone regions, domain-interface risks, or contact disruptions. The variant ranking stage 1112 may rank variants by foldability, stability, predicted function retention, structural plausibility, confidence, or combinations thereof. The engineering or screening output stage 1114 may provide one or more prioritized variants, one or more exclusion recommendations, one or more stabilization recommendations, one or more candidate therapeutic structures, one or more target-analysis outputs, or combinations thereof.

In some embodiments, the workflow 1102 is used for enzyme engineering. In some embodiments, the workflow 1102 is used for antibody engineering, therapeutic-protein engineering, peptide screening, target structure generation, mutation consequence analysis, personalized-medicine analysis, or combinations thereof. In some embodiments, the instability localization stage 1110 identifies regions associated with relatively high transformed gradient contribution, unfavorable transformed objective contribution, structural inconsistency, or reduced foldability score. In some embodiments, the engineering or screening output stage 1114 is used to guide experimental screening, wet-lab prioritization, variant selection, therapeutic design, or downstream computational studies.

FIG. 12 illustrates an example optional codon-derived, trajectory-based, strain-based, realization-cost-based, or Q_6-based embodiment 1202 for evaluating or constraining a protein-folding process. In the illustrated embodiment, the embodiment 1202 includes a codon-derived representation stage 1204, a trajectory representation stage 1206, a local strain evaluation stage 1208, a realization-cost evaluation stage 1210, and an optional conformation constraint or scoring output 1212. In some embodiments, the codon-derived representation stage 1204 maps one or more codons, residue states, symbolic states, or other discrete biological representations into one or more internal feature representations. In some embodiments, the trajectory representation stage 1206 represents one or more protein-folding paths, symbolic paths, state transitions, or structural progressions. In some embodiments, the local strain evaluation stage 1208 computes one or more strain-related quantities associated with one or more adjacent states, local transitions, local distances, or local realization relationships. In some embodiments, the realization-cost evaluation stage 1210 computes one or more realization or path costs associated with a candidate folding pathway or candidate final structure.

In some embodiments, one or more values generated by the embodiment 1202 are used as part of the nonnegative conformation-associated energy or cost E. In some embodiments, one or more values generated by the embodiment 1202 are used as additional constraints, regularizers, local terms, ranking terms, or validation terms. In some embodiments, folding is modeled as minimization of one or more trajectory-strain or realization-cost quantities over one or more codon-derived or Q_6-related representations. In some embodiments, the optional conformation constraint or scoring output 1212 is combined with one or more recognition-based energy values, one or more transformed force-field values, or one or more hybrid objective values. In some embodiments, this embodiment broadens the disclosed workflow to include additional representation families without requiring that every implementation use such representations.

The foregoing description is intended to illustrate and not limit the disclosed systems and methods. A protein, peptide, chain, domain, candidate conformation, predicted structure, native-like conformation, conformation-associated energy, transformed objective, optimizer, update step, convergence condition, foldability score, misfolding-prone region, validation metric, mutation variant, deployment environment, and accelerator may each be implemented in multiple ways consistent with the disclosure. One or more illustrated elements may be added, removed, reordered, repeated, combined, subdivided, parallelized, specialized, or generalized. One or more operations described in connection with one figure may be used with one or more operations described in connection with another figure. One or more embodiments may use all illustrated features, while other embodiments may use only a subset of the illustrated features. Accordingly, the disclosed subject matter includes variations, modifications, and equivalents falling within the scope of the appended claims.

What is claimed is:

1. A computer-implemented method for protein structure prediction, the method comprising:
receiving, by one or more processors, a protein-related input;
generating, by the one or more processors, one or more candidate conformations corresponding to the protein-related input;
computing, by the one or more processors, a nonnegative conformation-associated energy or cost for at least one of the one or more candidate conformations;
transforming, by the one or more processors, the nonnegative conformation-associated energy or cost using a recognition coverage function;
forming, by the one or more processors, a transformed conformational objective based at least in part on the transformed nonnegative conformation-associated energy or cost;
iteratively updating, by the one or more processors, the at least one candidate conformation using the transformed conformational objective; and
outputting, by the one or more processors, one or more predicted protein structures or one or more structure-related prediction results.

2. The method of claim 1, wherein the recognition coverage function is defined as:
$$F\_cov ( E , X\_opt ) = E / ( E + X\_opt ) ,$$
where E is the nonnegative conformation-associated energy or cost and X_opt is a scaling parameter.

3. The method of claim 2, wherein X_opt equals the golden ratio divided by pi.

4. The method of claim 1, wherein forming the transformed conformational objective comprises computing:
$$E\_RCF , i ( C ) = F\_cov ( E\_i ( C ) , X\_opt ) * ( Delta theta\_i ) \hat{} 2 ,$$
for one or more local structural components of a candidate conformation C, wherein DeltaTheta_i represents a torsion-angle deviation for a torsion group i.

5. The method of claim 4, wherein forming the transformed conformational objective further comprises computing:
$$E\_RCF ( C ) = sum over i of [ F\_cov ( E\_i ( C ) , X\_opt ) * ( Delta theta\_i ) \hat{} 2 ] .$$

6. The method of claim 1, wherein iteratively updating the at least one candidate conformation comprises computing an iteration-specific update parameter alpha_k according to: alpha_k=alpha_base*F_cov(E(C_k), X_opt), where alpha_base is a base update parameter and C_k is a candidate conformation at iteration k.

7. The method of claim 6, wherein iteratively updating the at least one candidate conformation further comprises computing: C_(k+1)=C_k-alpha_k*G_k, where G_k is a gradient, derivative, or directional update quantity derived from the transformed conformational objective.

8. The method of claim 1, wherein the nonnegative conformation-associated energy or cost comprises a recognition-based cost computed as a nonnegative mismatch between (i) contacts implied by a candidate conformation and (ii) a contact prior derived from the protein-related input, and wherein the recognition-based cost is aggregated across residue pairs using one or more weights.

9. The method of claim 1, wherein the nonnegative conformation-associated energy or cost comprises one or more bond terms, angle terms, torsion terms, steric terms, electrostatic terms, solvation terms, contact terms, residue-neighborhood terms, graph-based terms, strain terms, realization-cost terms, or combinations thereof.

10. The method of claim 1, wherein generating the one or more candidate conformations comprises generating random initial conformations, template-guided conformations, fragment-guided conformations, coarse-grained conformations, backbone-first conformations, mutation-local conformations, domain-wise conformations, or multiple restart conformations.

11. The method of claim 1, wherein iteratively updating the at least one candidate conformation comprises using gradient descent, projected gradient descent, coordinate descent, stochastic updates, momentum-based updates, quasi-Newton updates, trust-region updates, beam-assisted refinement, population-assisted refinement, simulated annealing, or a hybrid optimization workflow.

12. The method of claim 1, further comprising validating and ranking a plurality of candidate conformations based on one or more of RMSD, structural consistency, contact satisfaction, internal stability, foldability score, consensus across restarts, restraint satisfaction, or comparison against one or more reference structures.

13. The method of claim 1, wherein the one or more structure-related prediction results comprise one or more confidence values, one or more foldability metrics, one or more instability indicators, one or more misfolding-prone region indicators, one or more mutation rankings, or combinations thereof.

14. The method of claim 1, further comprising reducing an effective conformational search space using one or more contact constraints, residue-neighborhood restrictions, clash filters, graph restrictions, torsion-window restrictions, domain decomposition, beam truncation, mutation-local restrictions, or combinations thereof.

15. The method of claim 1, wherein the protein-related input comprises an amino-acid sequence, a FASTA-formatted sequence, a partial structure, one or more template structures, one or more structural restraints, one or more contact constraints, one or more mutation definitions, one or more domain definitions, one or more ligand or cofactor identifiers, or combinations thereof.

16. The method of claim 1, further comprising evaluating one or more stopping conditions and stopping the iterative updating when at least one stopping condition is satisfied, wherein the one or more stopping conditions include: (i) an objective improvement condition in which an absolute change in the transformed conformational objective is less than an improvement threshold epsilon for a specified number W of consecutive iterations, (ii) a gradient or directional-update magnitude condition in which a magnitude of the gradient, derivative, or directional update quantity is less than a threshold gamma, or (iii) an iteration budget or time budget is reached, wherein epsilon is selected from 10\hat{ }−6 to 10\hat{ }−2, W is selected from 5 to 200, and gamma is selected from 10\hat{ }−6 to 10\hat{ }−2.

17. The method of claim 1, wherein transforming the nonnegative conformation-associated energy or cost using the recognition coverage function comprises using a generalized recognition coverage function defined as:
$$F\_alt ( E , X\_opt , alpha ) = E \hat{} alpha / ( E \hat{} alpha + X\_opt \hat{} alpha ) ,$$
where alpha is a positive parameter.

18. The method of claim 1, further comprising computing one or more validation metrics for a plurality of candidate conformations and applying a decision rule that accepts a candidate structure when at least one validation metric is within an acceptance threshold band and otherwise performs a restart using a modified initialization, modified constraints, modified resolution level, modified base update parameter, or combinations thereof, wherein the acceptance threshold band comprises one or more of: RMSD≤T_RMSD when a reference is available, clash score≤T_clash, and contact satisfaction≥T_contact.

19. A system for protein structure prediction, comprising:
one or more processors; and
one or more memories storing instructions that, when executed by the one or more processors, cause the one or more processors to:
receive a protein-related input;
generate one or more candidate conformations corresponding to the protein-related input;
compute a nonnegative conformation-associated energy or cost for at least one of the one or more candidate conformations;
transform the nonnegative conformation-associated energy or cost using a recognition coverage function;
form a transformed conformational objective based at least in part on the transformed nonnegative conformation-associated energy or cost;
iteratively update the at least one candidate conformation using the transformed conformational objective; and
output one or more predicted protein structures or one or more structure-related prediction results.

20. A non-transitory computer-readable medium storing instructions that, when executed by one or more processors, cause the one or more processors to perform the method of claim 1.
